(19) United States
(12) Patent Application Publication
(10) Pub. No.: US 2025/0127989 A1
(43) Pub. Date: Apr. 24, 2025

(54) Medical Injections

(71) Applicant: Gilead Sciences, Inc., Foster City, CA (US)

(72) Inventors: Claudia Daniela Bautista Trevino, Foster City, CA (US); James Coates, Highfields Caldecote (GB); Rosemary Earl, Northstowe (GB); Will Harris, Newmarket (GB); Karl Hewson, Suffolk (GB); John Hiles, Cheshire (GB); Lewis Siu Tai Ho, St. Neots (GB); Manesh Patel, Milton (GB); Frances Anne Penrose, Ely (GB); Jenny Shepherd, East Sussex (GB)

(21) Appl. No.: 18/900,367

(22) Filed: Sep. 27, 2024

Related U.S. Application Data
(60) Provisional application No. 63/586,999, filed on Sep. 29, 2023.

(51) Int. Cl. A61M 5/20; A61M 5/32

(52) U.S. Cl. CPC A61M 5/2046; A61M 5/3216

(57) ABSTRACT

A medicament delivery device includes a housing, a container disposed within the housing and configured to contain medicament, a plunger slidably disposed within the container, a plunger rod configured to push the plunger through the container to dispense the medicament when the container contains medicament, and a gas canister configured to travel with the plunger rod and containing a compressed gas, wherein the gas canister is configured to release the compressed gas to drive the plunger rod to dispense the medicament out of a distal end of the container.

### CROSS REFERENCE TO RELATED APPLICATIONS

This application claims the benefit of U.S. Provisional Application No. 63/586,999, filed on Sep. 29, 2023, the entire contents of which is hereby incorporated by reference in its entirety.

### TECHNICAL FIELD

This disclosure relates to medical injections.

### BACKGROUND

An injection typically refers to the act of administering a liquid (e.g., a drug) into a patient's body tissue. Injecting medicament into a patient can allow the medicament to be absorbed relatively rapidly.

### SUMMARY

The present systems and methods can provide relatively high volume injections of relatively high viscosity medicaments. For example, in some embodiments, autoinjectors described herein provide a relatively easy to use device for injecting a volume of 5 milliliters (mL) of a medicament having a viscosity of 3000 centipoise (cP) without requiring a relatively large amount of effort by the user. In general, a gas canister included in the autoinjector provides an injection force sufficient to inject the medicament at the desired velocity to meet a desired delivery time. Using the gas canister to provide the large injection force reduces the force exerted by the user to sufficiently deliver an injection. In some embodiments, the gas canister is included in the plunger rod to reduce the volume of the autoinjector. Generally, an autoinjector with a reduced volume is more easily managed and wielded by the user than a bulkier autoinjector. In some embodiments, the autoinjector includes an interlock that prevents firing of the autoinjector until a desired time or action, e.g., when a needle is attached to the distal end of a syringe of the autoinjector.

In an embodiment, a medicament delivery device as provided herein includes a housing, a container disposed within the housing and configured to contain medicament, a plunger slidably disposed within the container, a plunger rod configured to push the plunger through the container to dispense the medicament when the container contains medicament, and a gas canister configured to travel with the plunger rod and containing a compressed gas, wherein the gas canister is configured to release the compressed gas to drive the plunger rod to dispense the medicament out of a distal end of the container.

Some embodiments provided herein incorporate one or more of the following features.

In some embodiments, the container contains 1-10 mL of medicament.

In some embodiments, the container contains 2-8 mL of medicament.

In some embodiments, the container contains 4-6 mL of medicament.

In some embodiments, the container contains 5 mL of medicament.

In some embodiments, the container contains 3-10 mL of medicament.

In some embodiments, the medicament has a viscosity within a range of 1-3000 cP.

In some embodiments, the medicament has a viscosity within a range of 1-2250 cP.

In some embodiments, the medicament has a viscosity within a range of 1750-2750 cP.

In some embodiments, the medicament has a viscosity within a range of 2000-2200 cP.

In some embodiments, the medicament has a viscosity of at least 2200 cP.

In some embodiments, the medicament delivery device includes a button configured to activate an element configured to pierce the gas canister, wherein the gas canister is configured to release the compressed gas upon being pierced.

In some embodiments, the medicament delivery device includes an interlock configured to prevent the element from piercing the gas canister while the interlock is in a locked position.

In some embodiments, the housing includes cut-outs configured to accept the interlock when the interlock is in an unlocked position.

In some embodiments, the cut-outs allow the interlock to flex radially when the interlock is in the unlocked position.

In some embodiments, the housing prevents the interlock from flexing radially when the interlock is in the locked position.

In some embodiments, the medicament delivery device includes a needle configured to be attached to a distal end of the container.

In some embodiments, the interlock is configured to move to an unlocked position when the needle is attached to the medicament delivery device.

In some embodiments, the needle is 20-21 gauge.

In some embodiments, the needle is less than 25 gauge.

In some embodiments, the needle is 18-25 gauge.

In some embodiments, the needle has a length of at least 1.5 inches.

In some embodiments, the needle has a length of 1.5-2 inches.

In some embodiments, the needle has a length of 0.75-2.25 inches.

In some embodiments, the medicament delivery device includes a needle guard configured to selectively cover the needle.

In some embodiments, the housing includes at least one vent hole.

In some embodiments, the gas canister is attached to the plunger rod.

In some embodiments, the gas canister is disposed within the plunger rod.

In another embodiment, provided herein is a method of delivering medicament contained in a container including piercing a gas canister to release a compressed gas to drive a plunger rod and dispense the medicament, wherein the gas canister is configured to travel with the plunger rod.

Some embodiments provided herein include one or more of the following features.

In some embodiments, piercing the gas canister includes pressing a button to activate an element configured to pierce the gas canister.

In some embodiments, the method includes attaching a needle to a distal end of the container.

In some embodiments, attaching the needle to the container moves an interlock from a locked position to an unlocked position, wherein the interlock prevents movement of the button in the locked position and allows movement of the button in the unlocked position.

In some embodiments, attaching the needle to the container includes screwing a needle hub onto a thread of a container holder.

In some embodiments, the method includes venting the compressed gas from a housing via one or more vent holes.

In some embodiments, the gas canister is attached to the plunger rod.

In some embodiments, the gas canister is disposed within the plunger rod.

In an embodiment, provided herein is a kit which includes a medicament delivery device according to any one of the embodiments described above. The kit also includes a needle.

In some embodiments, the needle is configured to attach to the medicament delivery device.

In some embodiments, the kit comprises a plurality of needles.

In some embodiments, the needles have different sizes.

In some embodiments, the needles have different shapes.

In some embodiments, the needles are configured to attach to the medicament delivery device.

The details of one or more embodiments of the subject matter described herein are set forth in the accompanying drawings and the description below. Other features, objects, and advantages of the subject matter will be apparent from the description and drawings, and from the claims.

### DESCRIPTION OF DRAWINGS

FIG. 1 illustrates an example autoinjector.

FIG. 2 illustrates the inner components of an example autoinjector.

FIG. 3 illustrates a gas canister disposed within a plunger.

FIGS. 4A-C illustrate an interlock to prevent accidental firing of an autoinjector.

FIG. 5 illustrates a needle attached to an autoinjector.

FIGS. 6A and 6B illustrate an unlocked interlock that allows firing of an autoinjector.

FIGS. 7A-C illustrate movement of an interlock to allow firing of an autoinjector.

FIGS. 8A and 8B illustrate firing of an autoinjector.

FIG. 9 illustrates a needle safety cap to cover a needle.

FIG. 10 is a table of delivery time vs. force for high viscosity medicaments.

Like reference numbers and designations in the various drawings indicate like elements.

### DETAILED DESCRIPTION

In some embodiments, the present systems and methods provided herein provide relatively high volume injections of relatively high viscosity medicaments and provide a relatively large injection force while reducing the force applied by a user to sufficiently deliver an injection. In some embodiments, the present systems and methods also prevent firing of an autoinjector until a desired time or action, e.g., when a needle is attached to the autoinjector.

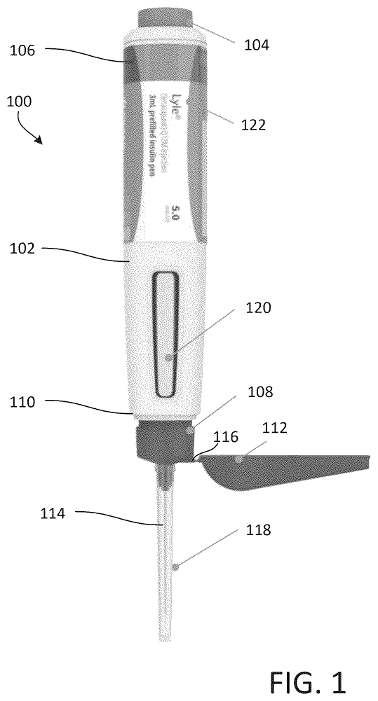

FIG. 1 illustrates an example autoinjector 100 including a housing 102, a button 104 on a proximal end 106 of the housing 102, and a needle hub 108 attached to a distal end 110 of the housing 102. The needle hub 108 includes a needle safety 112 that can selectively cover a needle 114 of the needle hub 108. For example, a user can bend the needle safety 112 about a hinge 116 to move the needle safety 112 over the needle 114. The needle 114 is covered by a needle cap 118, which a user removes prior to use of the autoinjector 100. In some embodiments, the button 104 is located elsewhere on the autoinjector. For example, the button 104 can be a side button that is located somewhere between the proximal end 106 and the distal end 110 of the housing 102. In some embodiments, the button is a lever that rotates relative to the housing 102 to activate the device. In some embodiments, the housing 102 includes flanges configured to accommodate the user's fingers.

The housing 102 includes a label 122 that provides information about the autoinjector 100. For example, in some embodiments, the label 122 includes medicament information, such as the type of medicament, the size of the dose, the delivery time of the dose, the expiration date of the medicament, a batch or lot number, information identifying the autoinjector 100 as a single use autoinjector, and company information. Optionally, the housing 102 does not include the label 122.

The housing 102 also includes a window 120, through which a user can see medicament contained within the autoinjector 100, e.g., within a container of the autoinjector 100 (see discussion below). In some embodiments, the window 120 helps a user determine whether the autoinjector 100 has been used. Before use of the autoinjector 100, the user can see through the window 120 to determine whether there is medicament within the autoinjector 100, for example, to determine that the autoinjector has not been used. In some embodiments, during use of the autoinjector 100, the user sees through the window 120 to determine whether the volume of medicament in the autoinjector 100 is decreasing. In some embodiments, after use of the autoinjector 100, the user sees through the window 120 to determine that there is no medicament in the autoinjector 100, for example, to determine that the autoinjector 100 has been used. In some embodiments, the user sees a plunger rod through the window 120 and determines that the autoinjector 100 has been used. In some embodiments, the user determines that the autoinjector has been used because the button 104 remains in a used position.

In some embodiments, the autoinjector 100 is used for intramuscular injections, which are injected into a muscle of the patient. Intramuscular injections can involve longer and/or thicker needles than subcutaneous injections, which are directed into fat tissue between the skin and muscle of a patient. As an example, in some embodiments, the needle 114 is 20 gauge and 1.5 inches long. As another example, in some embodiments, the needle 114 is 27 gauge and 0.5 inches long. Injecting medicament through a longer needle can involve a relatively large injection force, compared to injecting medicament through a shorter needle, for example when the medicament is a high volume, high viscosity medicament. Similarly, injecting medicament through a thinner needle can involve a relatively large injection force, as compared to injecting medicament through a wider needle. Intramuscular injections of high volume, high viscosity medicaments can be relatively difficult due, for example, to the relatively large injection forces and delivery times involved. In some embodiments, the autoinjector 100 (and other autoinjectors described herein) provides a sufficient injection force to inject a relatively high volume, relatively high viscosity medicament through a needle configured for intramuscular injections within the delivery time described below. The needle can have a variety of gauges (e.g., 20-21 gauge, less than 25 gauge, 18-25 gauge, etc.) for intramuscular injections. The needle can also have a variety of lengths (e.g., at least 1.5 inches, 1.5-2 inches, 1-2 inches, 0.75-2.25 inches, etc.) for intramuscular injections. In some embodiments, a kit includes the autoinjector 100 and a plurality of needles. The plurality of needles can be different sizes, different gauges, different shapes, etc., and a user can select a needle suitable for a specific injection. The user can attach the selected needle to the autoinjector 100 and use the autoinjector 100 as described herein.

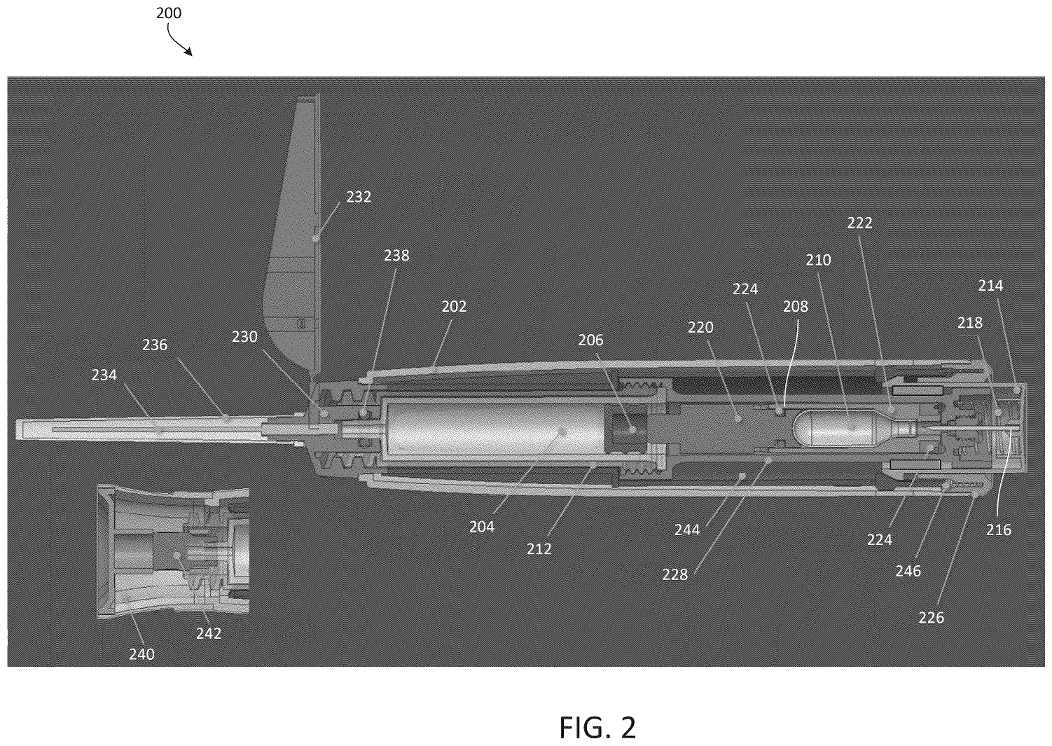

FIG. 2 illustrates an example autoinjector 200 that can provide a relatively large injection force to inject a relatively high volume, relatively high viscosity medicament through a needle 234 configured for intramuscular injections (e.g., similar to the autoinjector 100 of FIG. 1). The autoinjector 200 includes a housing 202, a container 204 retained within the housing 202, a plunger 206 slidably disposed within the container 204, a piston 208, and a gas canister 210. The container 204 is held in a container holder 212 connected to the housing 202. The container 204 can contain a medicament (not shown), and the autoinjector 200 can be activated by a user to dispense the medicament from the container 204. For example, a user can press a button 214 on the proximal end 226 of the housing 202 to pierce the gas canister 210, releasing compressed gas from within the gas canister 210. The compressed gas can include, for example, argon, carbon dioxide, krypton, xenon, etc. The button 214 is biased in the proximal direction by a button spring 218. The button 214 activates a firing pin 216 that pierces an end of the gas canister 210, as discussed further below. The compressed gas expands, driving the piston 208 against the plunger 206 and through the container 204. As the plunger 206 moves distally through the container 204, the medicament is dispensed through the needle 234. When the medicament is a relatively high viscosity medicament (e.g., 3000 cP), the plunger 206 can provide a relatively large force (e.g., 380 Newtons (N), which can be a time averaged force) to dispense the medicament from the needle 234 within a desired delivery time (e.g., under 50 seconds). Rapidly expanding gas (e.g., released from a gas canister 210) can provide the requisite force, e.g., for intramuscular injections. For example, in some embodiments, a rapidly expanding gas such as described with respect to the autoinjector 200 can provide a larger force than would be provided in an autoinjector that uses a drive spring.

As an example, in some embodiments, the container 204 has an internal volume to hold 5 mL of medicament. In some embodiments, the container 204 contains a variety of volumes of medicament (e.g., 1-10 mL of medicament, 2-8 mL of medicament, 4-6 mL of medicament, 3-10 mL of medicament, etc.).

In the autoinjector 200, the gas canister 210 is disposed within the piston 208. For example, the piston 208 includes a plunger rod portion 220 and a canister holder portion 222. The plunger rod portion 220 is configured to interact with the plunger 206 to drive the plunger 206 through the container 204. This interaction can include insertion of a distal end of the plunger rod portion 220 into a void defined within the plunger 206. The canister holder portion 222 provides a recess to receive the gas canister 210 within the canister holder portion 222. Seals 224 seal the connection between the plunger rod portion 220 and the canister holder portion 222 and seal the other end of the canister holder portion 222. When the gas canister 210 releases the compressed gas, the seals 224 prevent the released gas from exiting other portions of the piston 208. The released gas is directed proximally so that the force of the expanding gas drives the piston 208 distally to dispense medicament. A pressure vessel 228 connects to the container holder 212 and retains the piston 208 in alignment with the container 204. The released gas expands within the pressure vessel 228 to drive the piston 208 and the plunger 206 into the container 204.

Disposing the gas canister 210 within the piston 208 reduces the volume of the autoinjector 200. For example, if the gas canister was not disposed within the piston, the housing would need to be extended past the piston 208 at least the length of the gas canister 210 to provide sufficient space for the gas canister 210. In contrast, because the length of the gas canister 210 is incorporated into the piston 208, the housing 202 does not have to extend past the piston 208 the additional length of the gas canister 210. In some embodiments, the gas canister is partially (e.g., at least partially) disposed within the piston. Reducing the volume of the autoinjector 200 is advantageous because an autoinjector with a reduced volume is more easily managed and wielded by a user than a bulkier, larger autoinjector.

The autoinjector 200 also includes a needle hub 230 that detachably connects to the distal end of the container 204. The needle hub 230 includes a needle safety 232 that can selectively cover the needle 234 of the needle hub 230 (e.g., similar to the needle hub 108 of FIG. 1). The needle 234 is covered by a needle cap 236, which a user removes prior to use of the autoinjector 200. The needle hub 230 also includes a needle hub seal 238 that seals the connection between the needle hub 230 and the container 204. The needle hub seal 238 can prevent leaking between the container 204 and the needle hub 230.

In some embodiments, an autoinjector cap 240 is connected to a distal end of the housing 202 before the needle hub 230 is attached to the distal end of the container 204. For example, to use the autoinjector 200, a user can remove the autoinjector cap 240 from the distal end of the autoinjector 200 and then connect the needle hub 230 to the distal end of the container 204. The user can then press the button 214 to dispense the medicament, as discussed above. In some embodiments, the autoinjector cap 240 includes a luer tip cap 242 that seals the distal end of the container 204. The luer tip cap 242 reduces (e.g., prevents) leakage from the distal end of the container 204 prior to use of the autoinjector 200. In some embodiments, the luer tip cap 242 is configured to releasably attach to a corresponding luer tip disposed on a distal end of the container 204.

The autoinjector 200 can also include an interlock 244 to prevent the button 214 from being depressed before the needle hub 230 is attached to the container 204. The interactions between the interlock 244 and the button 214 will be described in greater detail below. Before use of the device, the interlock 244 is biased in the distal direction by an interlock spring 246 into a locked position. For example, the interlock spring 246 can be provided between the housing 202 and the interlock 244 to bias the interlock 244 in the distal direction. The interlock 244 prevents the button 214 from being depressed while the interlock 244 is in the locked position, as discussed further below. Since the interlock 244 prevents the button 214 from being depressed, the firing pin 216 is unable to pierce the gas canister 210 to dispense medicament. When the needle hub 230 is attached to the container 204, the needle hub 230 moves the interlock 244 against the bias of the interlock spring 246 into an unlocked position. The interlock 244 allows the button 214 to be depressed while the interlock 244 is in the unlocked position, as discussed further below. Since the interlock 244 allows the button 214 to be depressed (after the needle hub 230 is attached to the container), the firing pin 216 is able to pierce the gas canister 210 to dispense medicament through the needle hub 230.

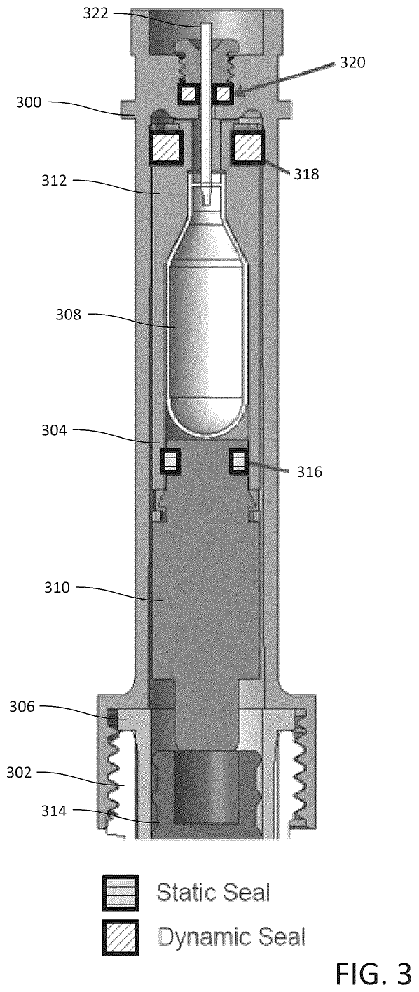

FIG. 3 illustrates an example of a pressure vessel 300. The pressure vessel 300 can be similar to the pressure vessel 228 of FIG. 2. The pressure vessel 300 connects to a container holder 302 to retain a piston 304 in alignment with a container 306. The piston 304 includes a gas canister 308 disposed within the piston 304. For example, the piston 304 includes a plunger rod portion 310 and a canister portion 312. The plunger rod portion 310 is configured to interact with the plunger 314 to drive the plunger 314 through the container 306. The canister portion 312 provides a recess to receive the gas canister 308 within the canister portion 312.

In some embodiments, the pressure vessel 300 includes one or more piston seals 316 that seal the connection between the plunger rod portion 310 and the canister portion 312. The piston seal 316 can be formed of, for example, nitrile butadiene rubber (e.g., having a 70 Shore A hardness). The piston seal 316 prevents gas released from the gas canister 308 from exiting via the connection between the plunger rod portion 310 and the canister portion 312. In some embodiments, the one or more piston seals 316 are static seals. For example, a static seal can create a seal between parts that do not move relative to one another. In some embodiments, no seals are between the plunger rod portion 310 and the canister portion 312. For example, the plunger rod portion 310 and the canister portion 312 can be formed of a single piece, such that there is no connection between the plunger rod portion 310 and the canister portion 312 for gas to exit. When the plunger rod portion 310 and the canister portion 312 are two connected pieces, one or more piston seals 316 can prevent gas from exiting via the connection between the plunger rod portion 310 and the canister portion 312.

In some embodiments, the pressure vessel 300 also includes one or more lip seals 318 that seal the area between the piston 304 and the pressure vessel 300. The lip seal 318 can be formed of, for example, nitrile butadiene rubber (e.g., having a 75 Shore A hardness, or an 85 Shore A hardness). The lip seal 318 prevents gas from escaping around the piston 304 and into the container 306. Keeping the expanding gas proximal to the piston 304 drives the piston 304 distally. If the gas is able to escape around the piston 304, the piston 304 may not be sufficiently driven distally to dispense medicament. The lip seal 318 can keep the expanding gas proximal to the piston 304 by sealing the area between the piston 304 and the pressure vessel 300. In some embodiments, the one or more lip seals 318 are dynamic seals. For example, a dynamic seal can create a seal between parts that move relative to one another.

In some embodiments, the pressure vessel 300 also includes one or more firing pin seals 320 that seal the area between the pressure vessel 300 and the firing pin 322 that pierces the gas canister 308. The firing pin seal 320 can be formed of, for example, an elastomer (e.g., a fluoroelastomer, such as Viton™, with a 70 Shore A hardness). The firing pin seal 320 can be formed of rubber. The firing pin seal 320 prevents gas from escaping through the back of the pressure vessel 300. Keeping the expanding gas within the pressure vessel 300 provides a defined volume for the gas to expand within. Providing a defined volume for the gas to expand within controls the force exerted on the piston 304 by the expanding gas. For example, if the expanding gas is not restricted to a defined volume, then the gas may not exert the desired injection force on the piston 304. The firing pin seal 320 can keep the expanding gas within the defined volume of the pressure vessel 300 by sealing the area between the pressure vessel 300 and the firing pin 322. In some embodiments, the one or more firing pin seals 320 are dynamic seals.

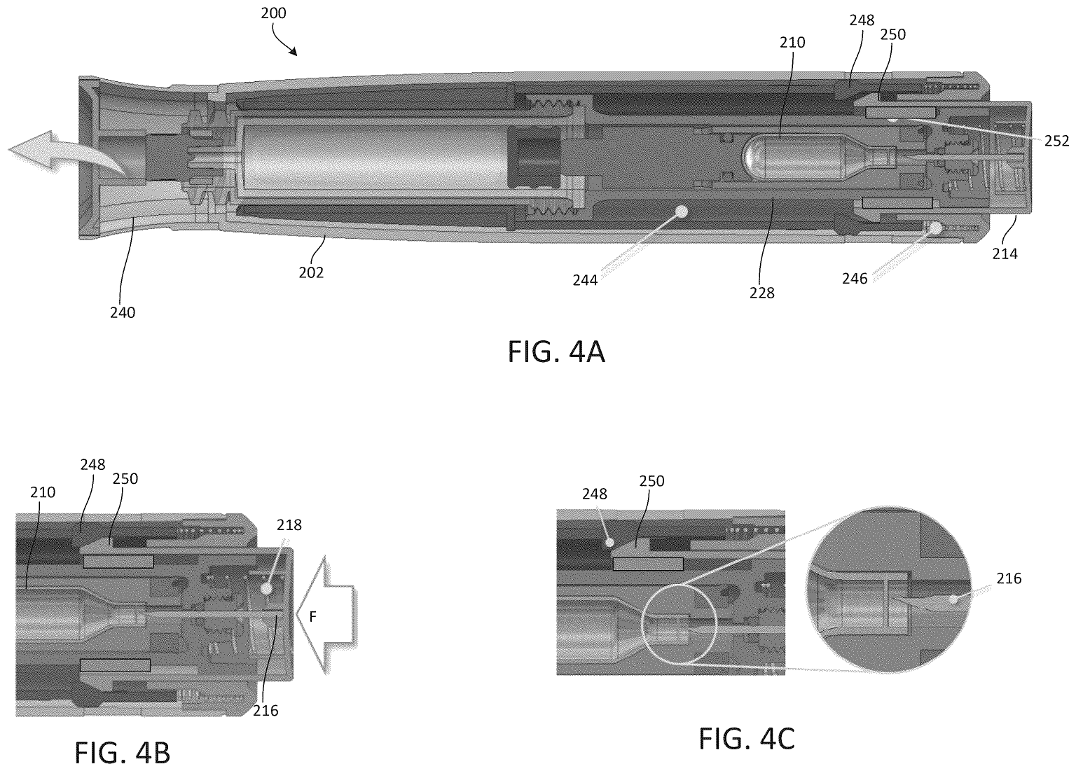

FIGS. 4A-C illustrate the autoinjector 200 with the interlock 244 in a locked position, and the cap 240 that is removed by the user prior to use of the autoinjector 200. Removing the cap 240 from the autoinjector 200 allows a needle hub (not shown) to be connected to the autoinjector 200, as discussed above.

The interlock 244 is biased in the distal direction by an interlock spring 246 into a locked position. For example, the interlock spring 246 can be provided between the housing 202 and the interlock 244 to bias the interlock 244 in the distal direction. The interlock 244 prevents the button 214 from being depressed while the interlock 244 is in the locked position. For example, the interlock 244 includes interlock snaps 248 that interact with button snaps 250 to prevent the button 214 from being depressed. When the user attempts to depress the button 214, e.g., as illustrated by the arrow F in FIG. 4B, the interlock snaps 248 interact with the button snaps 250 and prevent the button 214 from moving enough for the firing pin 216 to pierce the gas canister 210. For example, FIG. 4C illustrates a closer view of the firing pin 216 being unable to pierce the gas canister 210.

In some embodiments, the button 214 is able to move a relatively small distance (e.g., 2 mm) while the interlock 244 is in the locked position. The relatively small distance is still not sufficient for the firing pin 216 to pierce the gas canister 210. A button spring 218 can bias the button 214 in the proximal direction away from the interlock 244.

In some embodiments, the pressure vessel 228 includes protrusions 252 to prevent the button snaps 250 from biasing inwards away from the interlock snaps 248 and moving past the interlock snaps 248 while the interlock 244 is in the locked position. In some embodiments, the button snaps 250 are not flexible and cannot bias inwards, and the protrusions 252 are optional.

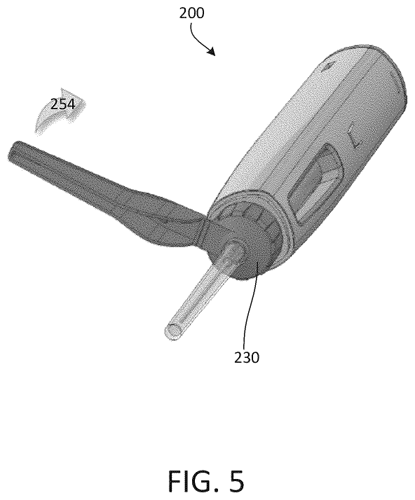

FIG. 5 illustrates the needle hub 230 being attached to an autoinjector 200. For example, a user can attach the needle hub 230 to the autoinjector 200. Attaching the needle hub 230 to the autoinjector 200 can move an interlock from the locked position to the unlocked position, as discussed above. In the illustrated example, the needle hub 230 is rotated to attach to the autoinjector 200, e.g., via a screw connection. In some embodiments, the needle hub 230 is attached to the autoinjector via other connections, e.g., bayonet connections, snap connections, etc. The needle hub 230 can connect to the container 204 of the autoinjector 200, as discussed above. The rotation of the needle hub 230 is illustrated by the arrow 254 in the clockwise direction. In some embodiments, the needle hub 230 is rotated 90° to attach the needle hub 230 to the autoinjector 200. The needle hub 230 can be designed so that the needle hub is rotated a variety of amounts (e.g., 180°, 270°, 360°, etc.) to attach the needle hub 230 to the autoinjector 200.

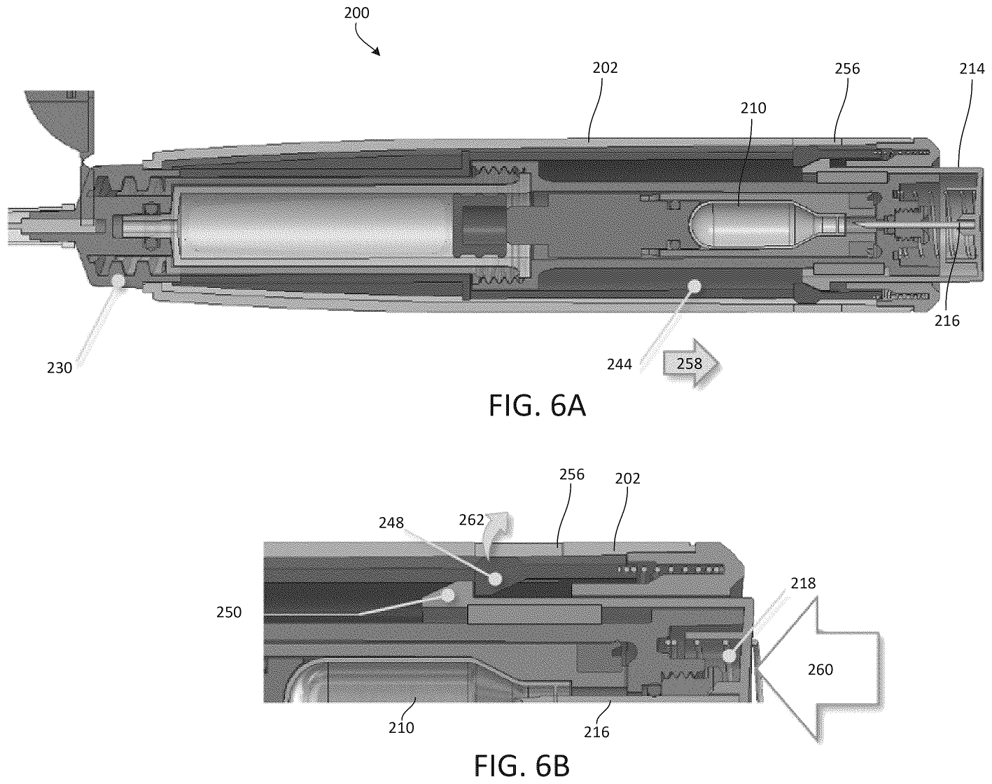

FIGS. 6A and 6B illustrate an autoinjector 200 with an interlock 244 that has moved to the unlocked position due to attachment of a needle hub 230. As discussed above, attaching a needle hub to an autoinjector can move an interlock proximally to an unlocked position. The movement of the interlock 244 in the proximal direction is illustrated by the arrow 258. When the interlock 244 is in the unlocked position, the button 214 can be depressed so that the firing pin 216 pierces the gas canister 210 to dispense the medicament. As illustrated in FIG. 6B, the user can depress the button 214 so that button snaps 250 of the button 214 move past interlock snaps 248 of the interlock. The depression of the button is illustrated by arrow 260. The button spring 218 depresses when the button 214 is depressed, and the button snaps 250 move past the interlock snaps 248. The button snaps 250 are able to move past the interlock snaps 248 because the interlock snaps 248 deflect radially outwards through cut-outs 256 in the housing 202. The radial deflection of the interlock snaps 248 is illustrated by the arrow 262. When the interlock 244 is moved proximally to the unlocked position, the interlock snaps 248 align with the cut-outs 256 in the housing 202 to allow the interlock snaps 248 to deflect outwards. When the interlock snaps 248 radially deflect outwards through the cut-outs 256, the button snaps 250 have space to move past the interlock snaps 248 so that the firing pin 216 can pierce the gas canister 210. For example, FIG. 6B illustrates the firing pin 216 piercing the gas canister 210. As discussed above, the expanding gas can provide a large injection force to inject a high volume, high viscosity medicament through a needle configured for intramuscular injections.

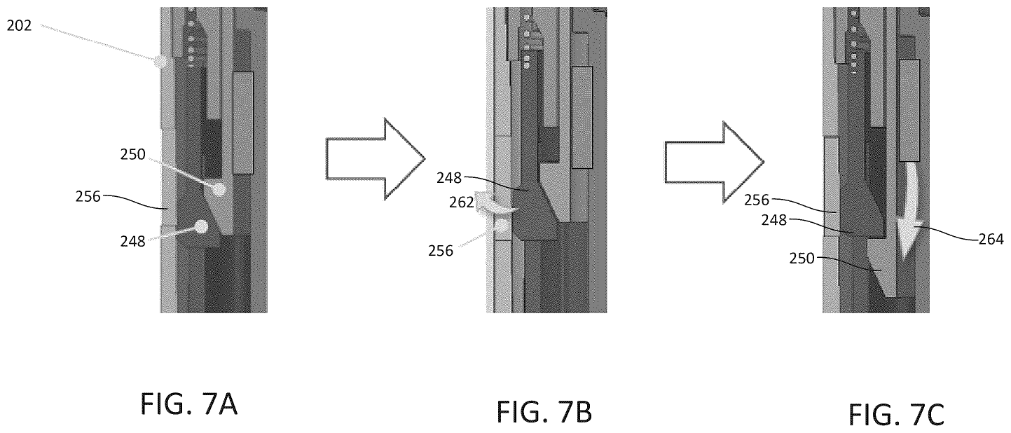

FIGS. 7A-C illustrate interlock snaps 248, the button snaps 250, and the cut-outs 256 in the housing 202 when the interlock 244 is in the locked position and when the interlock 244 is in the unlocked position. FIG. 7A illustrates the interlock snaps 248, the button snaps 250, and the cut-outs 256 when the interlock 244 is in the locked position, e.g., before the needle hub 230 is attached to the autoinjector 200. The interlock snaps 248 are not aligned with the cut-outs 256 in the housing 202 while the interlock 244 is in the locked position. Thus, the housing 202 prevents the interlock snaps 248 from deflecting radially outwards. Because the interlock snaps 248 cannot deflect radially outwards, the interlock snaps 248 prevent the button snaps 250 from moving distally.

FIG. 7B illustrates the interlock snaps 248, the button snaps 250, and the cut-outs 256 when the interlock 244 is in the unlocked position. For example, the interlock 244 moves proximally from the locked position to the unlocked position when the needle hub 230 is attached to the autoinjector 200, as discussed above. The proximal movement of the interlock 244 to the unlocked position aligns the interlock snaps 248 with the cut-outs 256 in the housing 202. When the interlock snaps 248 are aligned with the cut-outs 256, the interlock snaps 248 are able to deflect radially outwards. The ramped engagement between the button snaps 250 and the interlock snaps 248 cause the interlock snaps 248 to deflect radially outwards. The outward deflection of the interlock snaps 248 is illustrated by the arrow 262. The housing 202 does not prevent the interlock snaps 248 from deflecting radially outwards because the interlock snaps 248 are aligned with the cut-outs 256. Because the interlock snaps 248 can deflect radially outwards, the interlock snaps 248 do not prevent the button snaps 250 from moving distally.

FIG. 7C illustrates the interlock snaps 248, the button snaps 250, and the cut-outs 256 when the button snaps 250 move past the interlock snaps 248. The interlock snaps 248 are aligned with the cut-outs 256 because the needle hub 230 is connected to the autoinjector 200. The button snaps 250 are past the interlock snaps 248, and the movement of the button snaps 250 is illustrated by the arrow 264. The interlock snaps 248 are now proximal to the button snaps 250 due to the movement of the button snaps 250. The interlock snaps 248 prevent the button snaps 250 from moving proximally after injection starts. Preventing the button snaps 250 from moving proximally prevents the button 214 from moving proximally after injection begins. The engagement between the interlock snaps 248 and the button snaps 250 is not a ramped engagement, so the button snaps 250 do not cause the interlock snaps 248 to deflect radially outwards. When the button snaps move past the interlock snaps 248, the firing pin 216 pierces the gas canister 210, as discussed above. The expanding gas can provide a large injection force to inject a high volume, high viscosity medicament through a needle configured for intramuscular injections, as discussed above.

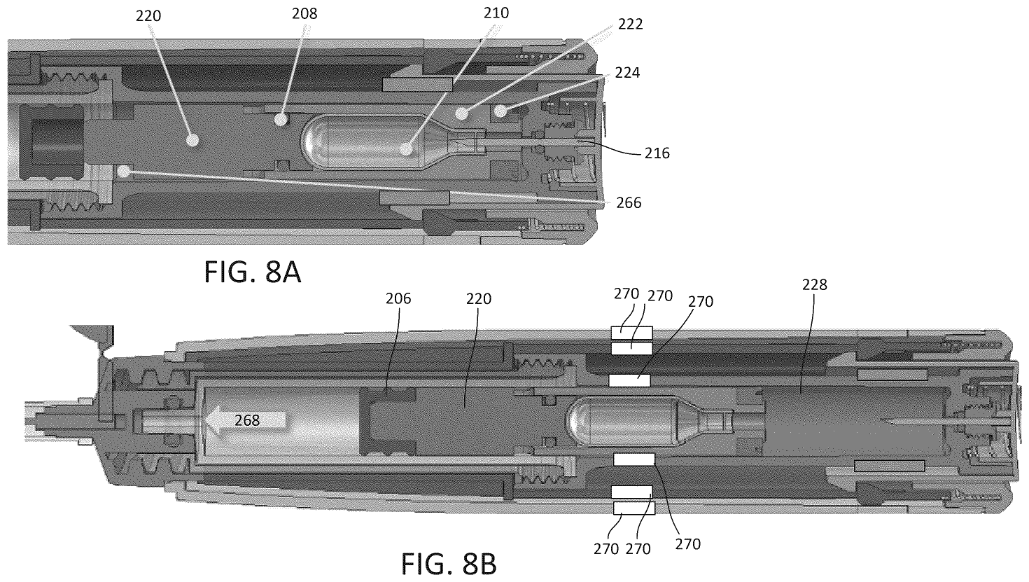

FIGS. 8A and 8B illustrate the autoinjector 200 dispensing medicament after the button snaps 250 move past the interlock snaps 248. The firing pin 216 pierces the gas canister 210, as discussed above and shown in FIG. 8A. Piercing the gas canister 210 causes the compressed gas to expand into the pressure vessel 228 to drive the piston 208 distally to dispense medicament, as discussed above. The rapid expansion of the compressed gas provides a sufficient injection force to inject a high volume, high viscosity liquid within a desired injection time. FIG. 8B illustrates movement of the piston 208 into the container 204 to dispense medicament. The movement of the piston 208 is represented by the arrow 268.

In some embodiments, the autoinjector 200 includes a start-of-dose click feature 266 that produces an audible sound when the firing pin 216 pierces the gas canister 210. For example, the click feature 266 can make an audible sound when the piston 208 compresses the click feature 266. Making an audible sound can be helpful, for example, to alert the user that the gas canister 210 has been pierced. In some embodiments, the click feature 266 can make an audible sound when the dose is fully dispensed. In some embodiments, the click feature 266 can click continuously during dispensing of the dose.

In some embodiments, the autoinjector 200 includes a number of vent holes 270 that allow the compressed gas to exit the autoinjector 200 after the dose is completely dispensed. For example, the pressure vessel 228, the interlock 244, and the housing 202 can include vent holes 270. The position of the vent holes 270 allows the compressed air to push the piston 208 through the container 204, and the vent holes 270 do not allow gas to escape during the dispensing. For example, the vent holes 270 are not accessible to the expanding gas until the piston has moved to an end-of-dose distal position. Until the piston 208 reaches the end-of-dose distal position, the gas cannot reach the vent holes 270.

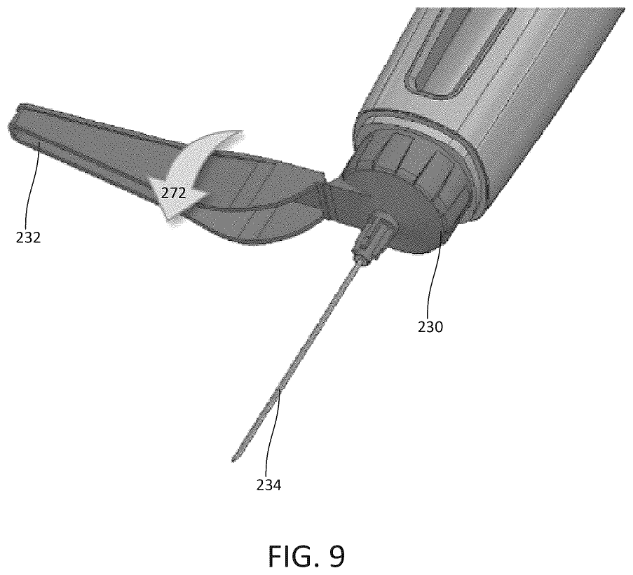

FIG. 9 illustrates the needle hub 230 after the dose has been dispensed, as described above. In some embodiments, the user can move the needle safety 232 to cover the needle 234 by bending the needle safety 232 about a hinge. The movement of the needle safety 232 over the needle 234 is represented by the arrow 272. Covering the needle 234 with the needle safety 232 can prevent accidental needle sticks after dispensing the dose.

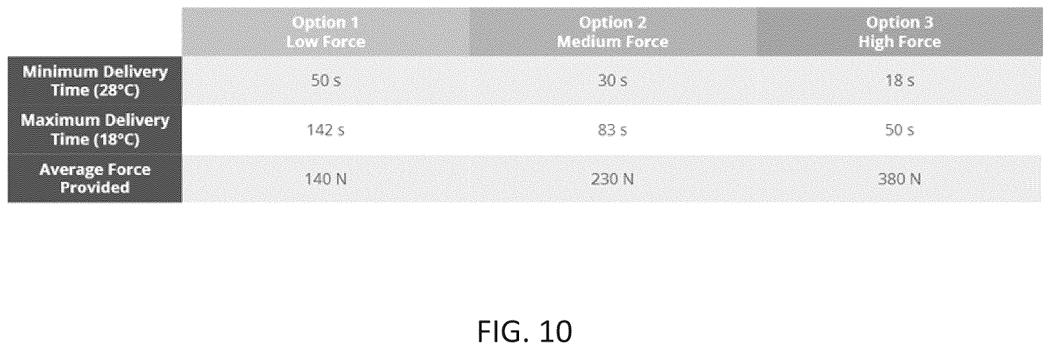

FIG. 10 illustrates a table of example forces and delivery times for delivering high volume, high viscosity medicaments using the autoinjector 200 discussed above. The high viscosity medicaments can have a viscosity of, e.g., 2250 cP. In some embodiments, the medicament can have a variety of viscosities (e.g., 1-3000 cP, 1-2250 cP, 1750-2750 cP, 2000-2500 cP, at least 2200 cP, etc.). The viscosity of the medicament can also change depending on the temperature of the medicament. For example, the medicament can have a higher viscosity at 18° C. than at 28° C. In one example, a medicament can have a viscosity of 2220 cP at 18° C., a viscosity of 1330 cP at 23° C., and a viscosity of 800 cP at 28° C. The force provided by the compressed gas can be designed to meet a desired delivery time for varying viscosities of medicament. The values in FIG. 10 are exemplary values for a medicament having a viscosity of 2220 cP at 18° C., a viscosity of 1330 cP at 23° C., and a viscosity of 800 cP at 28° C. The values were calculated for a needle length of 2 inches, a needle gauge of 20 gauge, a medicament volume of 5 mL, and a container radius of 5.93 mm. A relatively low injection force (e.g., an average force of 140 N) provides a maximum delivery time of 142 seconds (e.g., when the medicament has a viscosity of 2220 cP at 18° C.) and a minimum delivery time of 50 seconds (e.g., when the medicament has a viscosity of 800 cP at 28° C.). A relative medium injection force (e.g., an average force of 230 N) provides a maximum delivery time of 83 seconds (e.g., when the medicament has a viscosity of 2220 cP at 18° C.) and a minimum delivery time of 30 seconds (e.g., when the medicament has a viscosity of 800 cP at 28° C.). A relatively high injection force (e.g., an average force of 380 N) provides a maximum delivery time of 50 seconds (e.g., when the medicament has a viscosity of 2220 cP at 18° C.) and a minimum delivery time of 18 seconds (e.g., when the medicament has a viscosity of 800 cP at 28° C.). Thus, if a desired maximum delivery time is 50 seconds, the gas canister should exert an average injection force of 380 N to meet the desired delivery time.

A number of embodiments have been described. Nevertheless, it will be understood that various modifications may be made without departing from the spirit and scope of the disclosure. Accordingly, other embodiments are within the scope of the following claims.

1. A medicament delivery device, comprising:
a housing;
a container disposed within the housing and configured to contain medicament;
a plunger slidably disposed within the container;
a plunger rod configured to push the plunger through the container to dispense the medicament when the container contains medicament; and
a gas canister configured to travel with the plunger rod and containing a compressed gas, wherein the gas canister is configured to release the compressed gas to drive the plunger rod to dispense the medicament out of a distal end of the container.

2. The medicament delivery device of claim 1, wherein the container contains 1-10 mL of medicament.

3. The medicament delivery device of claim 1, wherein the container contains 2-8 mL of medicament.

4. The medicament delivery device of claim 1, wherein the container contains 4-6 mL of medicament.

5. The medicament delivery device of claim 1, wherein the container contains 5 mL of medicament.

6. The medicament delivery device of claim 1, wherein the container contains 3-10 mL of medicament.

7. The medicament delivery device of claim 1, wherein the medicament has a viscosity within a range of 1-3000 cP.

8. The medicament delivery device of claim 1, wherein the medicament has a viscosity within a range of 1-2250 cP.

9. The medicament delivery device of claim 1, wherein the medicament has a viscosity within a range of 1750-2750 cP.

10. The medicament delivery device of claim 1, wherein the medicament has a viscosity within a range of 2000-2200 cP.

11. The medicament delivery device of claim 1, wherein the medicament has a viscosity of at least 2200 cP.

12. The medicament delivery device of claim 1, further comprising a button configured to activate an element configured to pierce the gas canister, wherein the gas canister is configured to release the compressed gas upon being pierced.

13. The medicament delivery device of claim 1, further comprising an interlock configured to prevent the element from piercing the gas canister while the interlock is in a locked position.

14. The medicament delivery device of claim 13, wherein the housing comprises cut-outs configured to accept the interlock when the interlock is in an unlocked position.

15. The medicament delivery device of claim 14, wherein the cut-outs allow the interlock to flex radially when the interlock is in the unlocked position.

16. The medicament delivery device of claim 13, wherein the housing prevents the interlock from flexing radially when the interlock is in the locked position.

17. The medicament delivery device of claim 1, further comprising a needle configured to be attached to a distal end of the container.

18. The medicament delivery device of claim 17, wherein the interlock is configured to move to an unlocked position when the needle is attached to the medicament delivery device.

19. The medicament delivery device of claim 17, wherein the needle is 20-21 gauge.

20. The medicament delivery device of claim 17, wherein the needle is less than 25 gauge.

21. The medicament delivery device of claim 17, wherein the needle is 18-25 gauge.

22. The medicament delivery device of claim 17, wherein the needle has a length of at least 1.5 inches.

23. The medicament delivery device of claim 17, wherein the needle has a length of 1.5-2 inches.

24. The medicament delivery device of claim 17, wherein the needle has a length of 0.75-2.25 inches.

25. The medicament delivery device of claim 17, further comprising a needle guard configured to selectively cover the needle.

26. The medicament delivery device of claim 1, wherein the housing comprises at least one vent hole.

27. The medicament delivery device of claim 1, wherein the gas canister is attached to the plunger rod.

28. The medicament delivery device of claim 1, wherein the gas canister is disposed within the plunger rod.

29. A method of delivering medicament contained in a container, the method comprising:
piercing a gas canister to release a compressed gas to drive a plunger rod and dispense the medicament, wherein the gas canister is configured to travel with the plunger rod.

30. The method of claim 29, wherein piercing the gas canister comprises pressing a button to activate an element configured to pierce the gas canister.

31. The method of claim 29, further comprising attaching a needle to a distal end of the container.

32. The method of claim 31, wherein attaching the needle to the container moves an interlock from a locked position to an unlocked position, wherein the interlock prevents movement of the button in the locked position and allows movement of the button in the unlocked position.

33. The method of claim 31, wherein attaching the needle to the container comprises screwing a needle hub onto a thread of a container holder.

34. The method of claim 29, further comprising venting the compressed gas from a housing via one or more vent holes.

35. The method of claim 29, wherein the gas canister is attached to the plunger rod.

36. The method of claim 29, wherein the gas canister is disposed within the plunger rod.

37. A kit comprising:
a medicament delivery device according to claim 1; and
a needle.

38. The kit of claim 37, wherein the needle is configured to attach to the medicament delivery device.

39. The kit of claim 37, wherein the kit comprises a plurality of needles.

40. The kit of claim 39, wherein the needles have different sizes.

41. The kit of claim 39, wherein the needles have different shapes.

42. The kit of claim 39, wherein the needles are configured to attach to the medicament delivery device.
